(19) United States
(12) Patent Application Publication
(10) Pub. No.: US 2026/0144570 A1
(43) Pub. Date: May 28, 2026

(54) ANGLED SHEATH TIP

(71) Applicant: Boston Scientific Scimed, Inc., Maple Grove, MN (US)

(72) Inventors: Jackie Leung, Richmond Hill (CA); Matthew DiCicco, Toronto (CA); Alison Henstock, Milton (CA); Lauren Koon, Etobicoke (CA)

(21) Appl. No.: 19/397,052

(22) Filed: Nov. 21, 2025

Related U.S. Application Data
(60) Provisional application No. 63/724,626, filed on Nov. 25, 2024.

(51) Int. Cl. A61B 17/34; A61B 18/14; A61M 29/02; A61B 18/00

(52) U.S. Cl. CPC A61B 17/3417; A61B 18/1492; A61M 29/02; A61B 2018/00351; A61M 2029/025

(57) ABSTRACT

A transseptal crossing system for use in a transseptal crossing procedure includes an elongated puncturing device, a dilator, and a tubular outer sheath. The elongated puncturing device includes a distal tip configured for puncturing tissue. The dilator has a lumen configured for receiving the elongated puncturing device and a tapered distal portion configured to expand a puncture formed by the elongated puncturing device. The tubular outer sheath includes a proximal portion, an opposite sheath distal portion, and a sheath lumen extending between the proximal portion and the opposite sheath distal portion. The sheath lumen is configured for receiving the dilator. The sheath distal portion includes a distal end having an oblique profile such that a cross-sectional area of the distal end in contact with the tissue increases as the sheath is advanced over the dilator towards the tissue and through the tissue.

### CROSS REFERENCE TO RELATED APPLICATIONS

This application claims priority to U.S Provisional Patent Application No. 63/724,626 entitled “ANGLED SHEATH TIP,” filed Nov. 25, 2024, which is hereby incorporated by reference in its entirety.

### TECHNICAL FIELD

The present disclosure relates to medical systems and methods for creating channels in a tissue. More specifically, the present disclosure relates to medical systems and methods for performing transseptal crossing procedures.

### BACKGROUND

In transseptal crossing procedures, where access to the left atrium is achieved through transseptal puncture and crossing through the fossa ovalis, the step change in diameter between a dilator and sheath may result in an unsafe spike in force required to advance into the left atrium. This force can result in tissue damage. The step difference can be distributed over a longer length by angling the profile of the sheath tip, resulting in a smoother crossing requiring less force.

After puncture, there is still a risk of patient injury as the operator needs to advance a guiding sheath through the small opening. The opening is first expanded with a dilator but there is still a discrete change in diameter in the transition from the dilator to the sheath, and this can result in a buildup of mechanical force as the sheath is pushed against the septum. The transition from dilator to sheath carries a particularly higher risk because a significant portion of the dilator has already crossed into the left atrium so there is less space to accommodate for uncontrolled advancement of the assembly. Thus, this invention aims to reduce the force required to overcome this step change by designing a more gradual transition at the sheath tip. Physicians using this sheath will be able to access the right atrium without worrying about imparting an excessive amount of force to pass through the septum.

### SUMMARY

Example 1 is a system for use in a tissue crossing procedure. The system includes a tubular outer sheath includes a proximal portion and an opposite sheath distal portion. The sheath distal portion includes a distal end having an oblique profile such that a cross-sectional area of the distal end in contact with the tissue increases as the sheath is advanced towards the tissue and through the tissue.

Example 2 is the system of Example 1, further comprising a dilator having a tapered distal portion configured to expand a puncture formed in a tissue.

Example 3 is the system of any of Examples 1 or 2, wherein the oblique profile comprises a linearly angled profile extending between a leading edge of the distal end and a proximal edge of the distal end.

Example 4 is the system of any of Examples 1 or 2, wherein the oblique profile comprises a curved profile extending between a leading edge of the distal end and a proximal edge of the distal end.

Example 5 is the system of any of Examples 3 or 4, wherein the leading edge includes an apex, the proximal edge includes a vertex, and the apex and vertex are located on a common plane.

Example 6 is the system of any of Examples 1 or 2, wherein the oblique profile comprises a partial linearly angled profile extending between a leading edge of the distal end and a proximal edge of the distal end.

Example 7 is the system of Example 6, wherein the leading edge is perpendicular to a longitudinal axis extending through the tubular outer sheath.

Example 8 is the system of any of Examples 1 or 2, wherein the oblique profile comprises a wave profile.

Example 9 is the system of Example 8, wherein the wave profile includes at least one crest and at least one of trough.

Example 10 is the system of Example 9, wherein the at least one crest forms a leading edge of the distal end.

Example 11 is the system of any of Examples 9 or 10, wherein the at least one crest and the at least one trough are aligned at a common longitudinal location.

Example 12 is the system of any of Examples 9 or 10, wherein the at least one crest includes a plurality of crests and the at least one trough includes a plurality of troughs, and the plurality of crests or the plurality of troughs are aligned at different longitudinal locations.

Example 13 is the system of any of Examples 1-12, wherein the tubular outer sheath includes one or more markers located in the sheath distal portion.

Example 14 is the system of any of Examples 1-13, wherein the tubular outer sheath includes one or more openings located in the sheath distal portion.

Example 15 is the system of any of Examples 3-14, wherein the system includes a curved configuration having an inner radius and the leading edge is aligned with the inner radius such that the dilator bends into the leading edge.

Example 16 is a system for use in a tissue crossing procedure. The system includes an elongated puncturing device having a distal tip configured for puncturing tissue. The system includes a dilator having a lumen configured for receiving the elongated puncturing device and a tapered distal portion configured to expand a puncture formed by the elongated puncturing device. The system includes a tubular outer sheath having a proximal portion, an opposite sheath distal portion, and a sheath lumen extending between the proximal portion and the opposite sheath distal portion. The sheath lumen is configured for receiving the dilator. The sheath distal portion includes a distal end having an oblique profile such that a cross-sectional area of the distal end in contact with the tissue increases as the sheath is advanced over the dilator towards the tissue and through the tissue.

Example 17 is the system of Example 16, wherein the oblique profile comprises a linearly angled profile extending between a leading edge of the distal end and a proximal edge of the distal end.

Example 18 is the system of Example 16, wherein the oblique profile comprises a curved profile extending between a leading edge of the distal end and a proximal edge of the distal end.

Example 19 is the system of Example 17, wherein the leading edge includes an apex, the proximal edge includes a vertex, and the apex and vertex are located on a common plane.

Example 20 is the system of Example 16, wherein the oblique profile comprises a partial linearly angled profile extending between a leading edge of the distal end and a proximal edge of the distal end.

Example 21 is the system of Example 20, wherein the leading edge is perpendicular to a longitudinal axis extending through the tubular outer sheath.

Example 22 is the system of Example 16, wherein the oblique profile comprises a wave profile.

Example 23 is the system of Example 22, wherein the wave profile includes at least one crest and at least one of trough.

Example 24 is the system of Example 23, wherein the at least one crest forms a leading edge of the distal end.

Example 25 is the system of Example 23, wherein the at least one crest and the at least one trough are aligned at a common longitudinal location.

Example 26 is the system of Example 23, wherein the at least one crest includes a plurality of crests and the at least one trough includes a plurality of troughs, and the plurality of crests or the plurality of troughs are aligned at different longitudinal locations.

Example 27 is the system of Example 16, wherein the tubular outer sheath includes one or more markers located in the sheath distal portion.

Example 28 is the system of Example 16, wherein the tubular outer sheath includes one or more openings located in the sheath distal portion.

Example 29 is the system of Example 16, wherein the system includes a curved configuration having an inner radius.

Example 30 is the system of Example 29, wherein a leading edge of the distal end is aligned with the inner radius such that the dilator bends into the leading edge.

Example 31 is a system for use in a tissue crossing procedure. The system includes an elongated puncturing device having an energy emitting distal tip configured for puncturing tissue. The system includes a dilator having a lumen configured for receiving the elongated puncturing device and a tapered distal portion configured to expand a puncture formed by the elongated puncturing device. The system includes a tubular outer sheath having a proximal portion, an opposite sheath distal portion, and a sheath lumen extending between the proximal portion and the opposite sheath distal portion. The sheath lumen is configured for receiving the dilator. The sheath distal portion includes a distal end having an oblique profile such that a cross-sectional area of the distal end in contact with the tissue increases as the sheath is advanced over the dilator towards the tissue and through the tissue.

Example 32 is the system of Example 31, wherein the system includes a curved configuration having an inner radius.

Example 33 is the system of Example 32, wherein a leading edge of the distal end is aligned with the inner radius such that the dilator bends into the leading edge.

Example 34 is the system of Example 31, wherein the oblique profile comprises a linearly angled profile, a curved profile, a wave profile, or a partial linear angled profile.

Example 35 is a system for use in a tissue crossing procedure. The system includes an elongated puncturing device having a distal tip configured for puncturing tissue. The system includes a dilator having a lumen configured for receiving the elongated puncturing device and a tapered distal portion configured to expand a puncture formed by the elongated puncturing device. The system includes a tubular outer sheath having a proximal portion, an opposite sheath distal portion, and a sheath lumen extending between the proximal portion and the opposite sheath distal portion. The sheath lumen is configured for receiving the dilator. The sheath distal portion includes a distal end having an oblique profile such that a cross-sectional area of the distal end in contact with the tissue increases as the sheath is advanced over the dilator towards the tissue and through the tissue. The system includes a curved configuration having an inner radius. A leading edge of the distal end is aligned with the inner radius.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIGS. 1A-1C are schematic illustrations of a medical procedure within a patient’s heart utilizing a transseptal access system, in accordance with embodiments of the disclosure.

FIG. 2 illustrates a comparison between transseptal access system having a sheath with a tip perpendicular to a longitudinal axis of the sheath and a transseptal access system having a sheath with a tip including an oblique profile relative to a longitudinal axis of the sheath, in accordance with embodiments of the disclosure.

FIG. 3A is a side view of a transseptal access system having a sheath with an end including a linearly angled profile relative to a longitudinal axis of the sheath, in accordance with embodiments of the disclosure.

FIG. 3B is a top view of a distal end of the sheath of FIG. 3A, in accordance with embodiments of the disclosure.

FIG. 3C is a side view of the transseptal access system of FIG. 3A in a curved configuration during a transseptal crossing procedure, in accordance with embodiments of the disclosure.

FIGS. 4A-4D are cross-sectional views along portions of the transseptal access system of FIG. 3A, in accordance with embodiments of the disclosure.

FIG. 5 is a side view of the transseptal access system having a sheath with an end including a partial linearly angled profile relative to a longitudinal axis of the sheath, in accordance with embodiments of the disclosure.

FIG. 6 is a side view of the transseptal access system having a sheath with an end including a curved angled profile relative to a longitudinal axis of the sheath, in accordance with embodiments of the disclosure.

FIG. 7 is a side view of the transseptal access system having a sheath with an end including a wave profile relative to a longitudinal axis of the sheath, in accordance with embodiments of the disclosure.

FIG. 8A illustrates a first arrangement for a wave profile, in accordance with embodiments of the disclosure.

FIG. 8B illustrates a second arrangement for a wave profile, in accordance with embodiments of the disclosure.

FIG. 9 illustrates peak crossing forces for various arrangements of sheaths with an end including a linearly angled profile, in accordance with embodiments of the disclosure.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) the features in a given example used across all examples. Thus, no one figure should be interpreted as having any dependency or requirement related to any single component or combination of components illustrated therein. Additionally, various components depicted in a given figure may be, in examples, integrated with various ones of the other components depicted therein (and/or components not illustrated), all of which are considered to be within the ambit of the present disclosure.

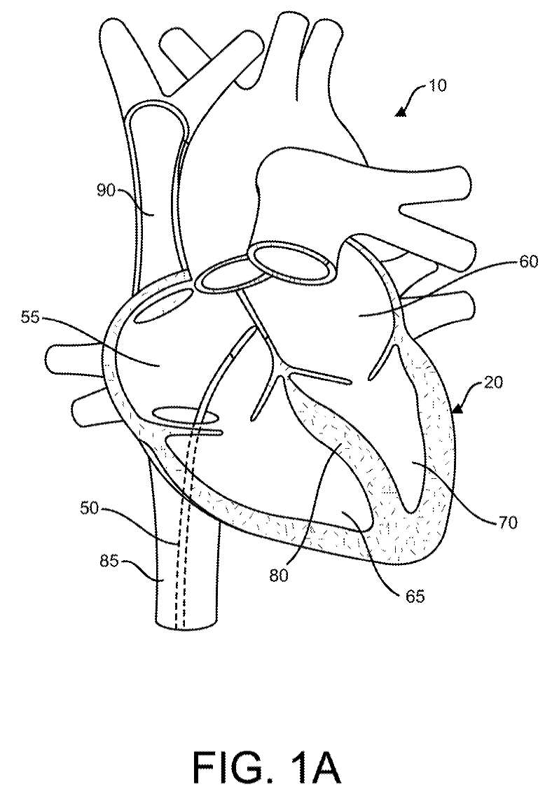

FIGS. 1A-1C are schematic illustrations of a medical procedure 10 within a patient’s heart 20 utilizing a transseptal access system 50 according to embodiments of the disclosure. As is known, the human heart 20 has four chambers, a right atrium 55, a left atrium 60, a right ventricle 65 and a left ventricle 70. Separating the right atrium 55 and the left atrium 60 is an atrial septum 75 and separating the right ventricle 65 and the left ventricle 70 is a ventricular septum 80. As is further known, deoxygenated blood from the patient’s body is returned to the right atrium 55 via an inferior vena cava (IVC) 85 or a superior vena cava (SVC) 90.

Various medical procedures have been developed for diagnosing or treating physiological ailments originating within the left atrium 60 and associated structures. Exemplary such procedures include, without limitation, deployment of diagnostic or mapping catheters within the left atrium 60 for use in generating electroanatomical maps or diagnostic images thereof. Other exemplary procedures include endocardial catheter-based ablation (e.g., radiofrequency ablation, pulsed field ablation, cryoablation, laser ablation, high frequency ultrasound ablation, and the like) of target sites within the chamber or adjacent vessels (e.g., the pulmonary veins and their ostia) to terminate cardiac arrythmias such as atrial fibrillation and atrial flutter. Still other exemplary procedures may include deployment of left atrial appendage (LAA) closure devices. Of course, the foregoing examples of procedures within the left atrium 60 are merely illustrative and in no way limiting with respect to the present disclosure.

The medical procedure 10 illustrated in FIGS. 1A-1C is an exemplary embodiment for providing access to the left atrium 60 using the transseptal access system 50 for subsequent deployment of the aforementioned diagnostic and/or therapeutic devices within the left atrium 60. As shown in FIGS. 1A-1C, target tissue site can be defined by tissue on the atrial septum 75. In the illustrated embodiment, the target site is accessed via the IVC 85, for example through the femoral vein, according to conventional catheterization techniques. In other embodiments, access to the target site on the atrial septum 75 may be accomplished using a superior approach wherein the transseptal access system 50 is advanced into the right atrium 55 via the SVC 90.

In the illustrated embodiment, the transseptal access system 50 includes an introducer sheath 100, a dilator 105 having a dilator body 107 and a tapered distal tip portion 108, and a radiofrequency (RF) perforation device 110, also known as a piercing device or puncturing device, having distal end portion 112 terminating in a tip electrode 115. As shown, in the assembled use state illustrated in FIGS. 1A-1C, the RF perforation device 110 can be disposed within the dilator 105, which itself can be disposed within the sheath 100. In one embodiment in which the transseptal access system 50 is deployed into the right atrium 55 via the IVC 85, a user introduces a guidewire (not shown) into a femoral vein, typically the right femoral vein, and advances it towards the heart 20. The sheath 100 may then be introduced into the femoral vein over the guidewire, and advanced towards the heart 20. In one embodiment, the distal ends of the guidewire and sheath 100 are then positioned in the SVC 90. These steps may be performed with the aid of an imaging system, e.g., fluoroscopy or ultrasonic imaging. The dilator 105 may then be introduced into the sheath 100 and over the guidewire, and advanced through the sheath 100 into the SVC 90. Alternatively, the dilator 105 may be fully inserted into the sheath 100 prior to entering the body, and both may be advanced simultaneously towards the heart 20. When the guidewire, sheath 100, and dilator 105 have been positioned in the superior vena cava, the guidewire is removed from the body, and the sheath 100 and the dilator 105 are retracted so that their distal ends are positioned in the right atrium 55. The RF perforation device 110 described can then be introduced into the dilator 105, and advanced toward the heart 20. In some aspects, the guidewire may itself include an RF electrode so as to function as an RF perforation device.

Subsequently, the user may position the distal end of the dilator 105 against the atrial septum 75, which can be done under imaging guidance. The RF perforation device 110 is then positioned such that electrode 115 is aligned with or protruding slightly from the distal end of the dilator 105. The dilator 105 and the RF perforation device 110 may be dragged along the atrial septum 75 and positioned, for example against the fossa ovalis of the atrial septum 75 under imaging guidance. A variety of additional steps may be performed, such as measuring one or more properties of the target site, for example an electrogram or ECG (electrocardiogram) tracing and/or a pressure measurement, or delivering material to the target site, for example delivering a contrast agent. Such steps may facilitate the localization of the tip electrode 115 at the desired target site. In addition, tactile feedback provided by medical RF perforation device 110 is usable to facilitate positioning of the tip electrode 115 at the desired target site.

With the tip electrode 115 and dilator 105 positioned at the target site, energy is delivered from an energy source, e.g., an RF generator, through the RF perforation device 110 to the tip electrode 115 and the target site. In some embodiments, the energy is delivered at a power of at least about 5 W at a voltage of at least about 200 V RMS (565 V peak-to-peak), and functions to vaporize cells in the vicinity of the tip electrode 115, thereby creating a void or perforation through the tissue at the target site. The user then applies force to the RF perforation device 110 so as to advance the tip electrode 115 at least partially through the perforation. In these embodiments, when the tip electrode 115 has passed through the target tissue, that is, when it has reached the left atrium 60, energy delivery is stopped. In some embodiments, the step of delivering energy occurs over a period of between about 1 s and about 5 s.

With the tip electrode 115 of the RF perforation device 110 having crossed the atrial septum 75, the dilator 105 can be advanced forward, with the tapered distal tip portion 108 operating to gradually enlarge the perforation to permit advancement of the distal end of the sheath 100 into the left atrium 60.

In some embodiments, the distal end portion 112 of the RF perforation device 110 is pre-formed to assume an atraumatic shape such as a J-shape (as shown in FIGS. 1B-1C), a pigtail shape or other shape selected to direct the tip electrode 115 away from the endocardial surfaces of the left atrium 60. Examples of such RF perforation devices can be found, for example, in U.S. Patent Application Nos. 16/445,790 and 16/346,404 assigned to Baylis Medical Company, Inc. The aforementioned pre-formed shapes can advantageously function to minimize the risk of unintended contact between the tip electrode 115 and tissue within the left atrium 60 and can also operate to anchor the distal end portion 112 within the left atrium 60 during subsequent procedural steps. For example, in embodiments, the RF perforation device 110 can be structurally configured to function as a delivery rail for deployment of a relatively larger bore therapy delivery sheath and associated dilator(s). In such embodiments, the dilator 105 and the sheath 100 are withdrawn following deployment of the distal end portion 112 of the RF perforation device 110 into the left atrium 60. The anchoring function of the pre-formed distal end portion 112 inhibits unintended retraction of the distal end portion 112, and corresponding loss of access to the perforated site on the atrial septum 75, during such withdrawal.

The transseptal access system 50 may be configured to achieve a plurality of different curvatures. This is useful to allow introduction into and positioning of the system 50 at a desired location within the heart 20. For example, the various curvatures allow for achieving desired positioning of the dilator 105 and the RF perforation device 110 along a portion of the atrial septum 75.

In some aspects, the RF perforation device 110 is replaced with a mechanical piercing device such as a needle having a sharp distal tip. In certain embodiments, the needle is configured to pierce the atrial septum 75 when the sharp distal tip is positioned on the atrial septum 75 and pressure is applied to the proximal end.

In some aspects, it may be desirable for the dilator 105, sheath 100, or RF perforation device 110 to include one or more surface electrodes. In various embodiments, thee one or more surface electrodes are located on a distal portion of the dilator 105, sheath 100, or RF perforation device 110 for use in ablation, mapping, pacing, or sensing a parameter within a portion of the heart 20. The one or more surface electrode may be connected to an electroanatomical mapping (EAM) system, generator, or other diagnostic system.

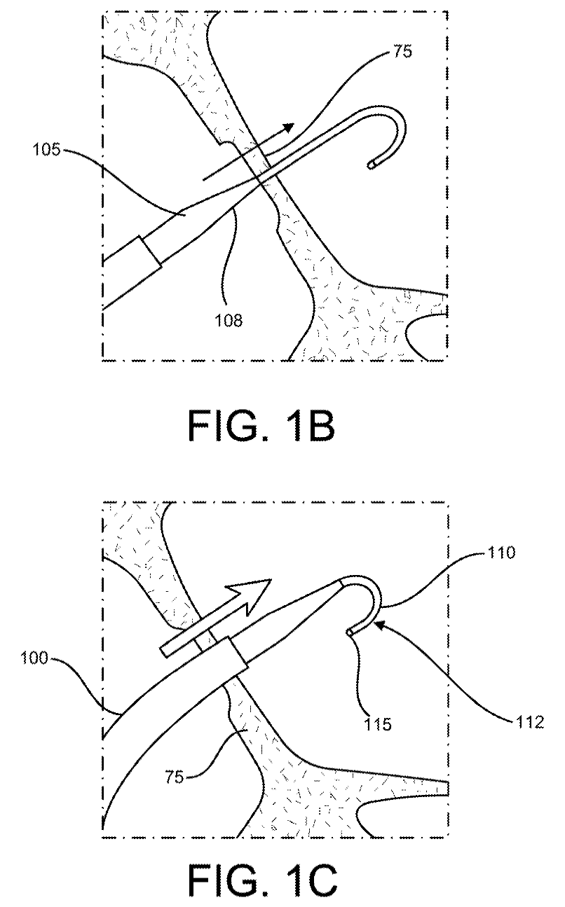

FIG. 2 illustrates a comparison between a transseptal access system 200 having an outer sheath 202 with a tip 204 perpendicular to a longitudinal axis of the sheath 202 and a transseptal access system 200’ having an outer sheath 202’ with an end 204’ including an oblique profile 206’ relative to a longitudinal axis of the sheath, in accordance with embodiments of the disclosure. As illustrated in the graph 210 comparing a position to the cross-sectional area of the outer sheath 202 in contact with tissue, there is an abrupt change in cross-sectional area in contact with the tissue as the tip 204 first contacts the tissue. This corresponds to an increase in force to pass the sheath 202 through the tissue. Conversely, as illustrated in the graph 210’, there is a more gradual increase in cross-sectional area of the sheath 202’ that contacts the tissue as the sheath 202’ is advanced. This more gradual increase correlates with a smoother force transition during a crossing procedure.

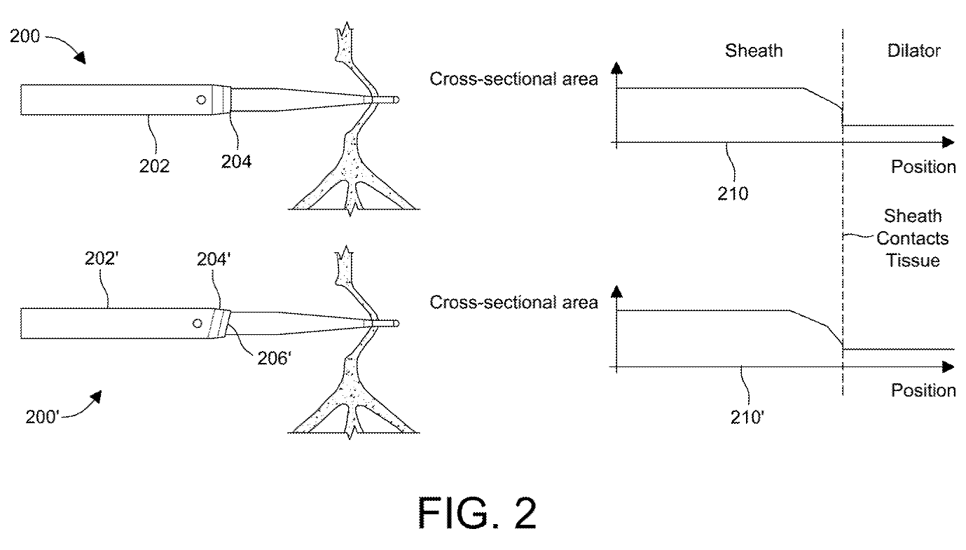

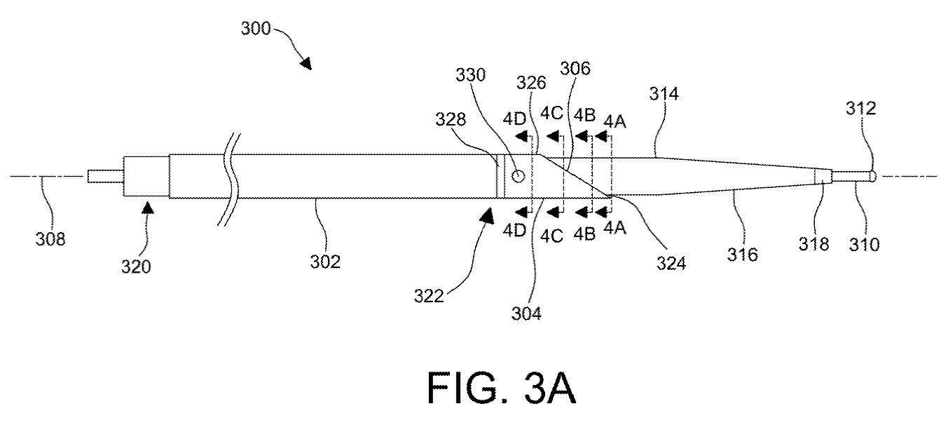

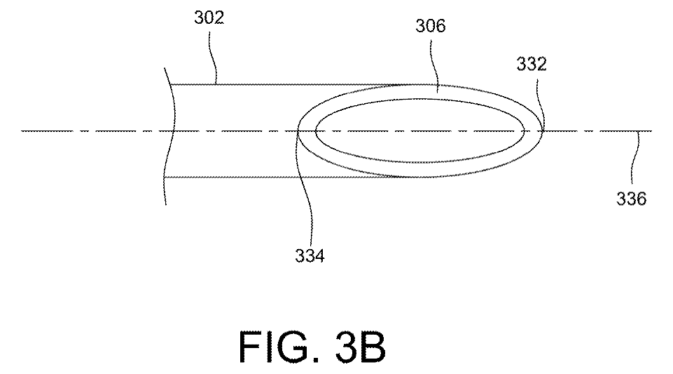

FIG. 3A is a side view of a transseptal access system 300 having a sheath 302 with a distal end 304 including an oblique profile 306 relative to a longitudinal axis 308 of the sheath 302, in accordance with embodiments of the disclosure. The system 300 includes an elongated puncturing device 310 having an energy emitting distal tip 312 configured for puncturing tissue. In some embodiments, the energy emitting distal tip 312 includes an electrode configured to deliver RF energy to a target tissue location for puncturing.

The system 300 includes a dilator 314 having a tapered distal portion 316. The dilator 314 includes a lumen configured to receive the elongated puncturing device 310. The dilator 314 is configured to expand a puncture formed by the elongated puncturing device 310. The tapered distal portion 316 gradually expands the puncture upon advancement into the tissue. The dilator 314 includes a marker 318 along the tapered distal portion 316. The marker 318 is formed of a material that increases visualization of the marker 318 using imaging and is configured to aid a user in navigating the system 300 to a desired tissue site. In some embodiments, the marker 318 is formed of a radiopaque material. In some embodiments, the marker 318 is formed of a material that enhances echogenic properties of the marker 318.

The tubular outer sheath 302 includes a proximal portion 320 and an opposite distal portion 322. The tubular outer sheath 302 includes a lumen configured to receive the dilator 314. The distal portion 322 includes the distal end 304 having an oblique profile 306 such that a cross-sectional area of the distal end 304 in contact with a tissue increases as the sheath 302 is advanced towards the tissue and through the tissue. FIG. 3A illustrates the oblique profile 306 including a linearly angled profile extending between a leading edge 324 of the distal end 304 and a proximal edge 326 of the distal end 304. The linearly angled profile is angled from the leading edge 324 to the proximal edge 326. In some embodiments, the linearly angled profile is angled between 10-degrees and 85-degrees relative to the longitudinal axis 308.

FIG. 3B is a top view of the distal end 304 of the tubular outer sheath 302. When viewed from the top, the oblique profile 306 appears generally as an ellipse or an oval. In some embodiments, the leading edge 324 includes a most distal point, an apex 332, and the proximal edge 326 includes a most proximal point, a vertex 334. In some embodiments, the apex 332 and the vertex 334 are located on a common plane 336. When viewed from the side, the oblique profile 306 appears linearly from the apex 332 to the vertex 332.

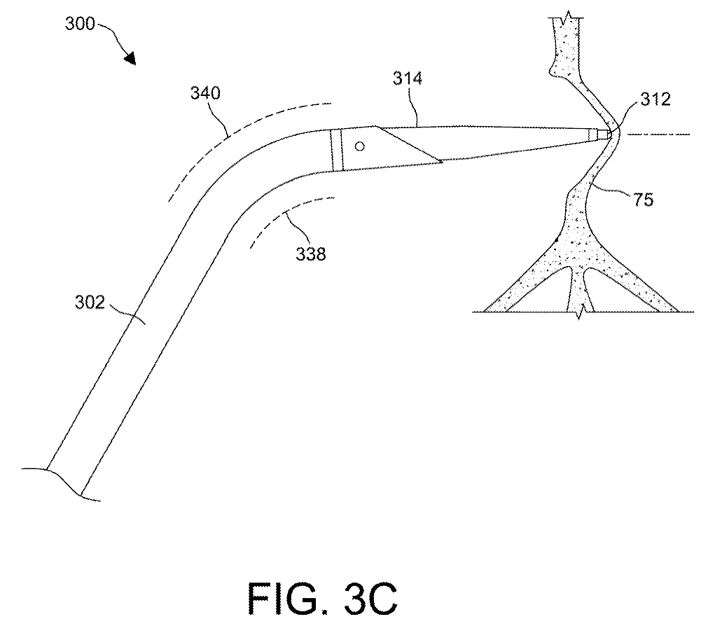

FIGS. 4A-4D are cross-sectional views along portions of the transseptal access system of FIG. 3A illustrating the change in cross-sectional area of the oblique profile 306 from the leading edge 324 towards the proximal edge 326. FIG. 4A illustrates the leading edge 324 of the oblique profile 306. FIG. 4B illustrates the oblique profile 306 increasing in area moving towards the proximal edge 326. FIG. 4C illustrates the oblique profile 306 further increasing in area moving towards the proximal edge 326. In FIG. 4D, the proximal edge 326 of the oblique profile 306 is shown. At the proximal edge 326 the oblique profile 306 transitions to the entire circumference of the sheath 302. Proximal of the proximal edge 326 the sheath includes a substantially constant cross-sectional area.

Referring again to FIG. 3A, the tubular outer sheath 302 includes one or more markers 328 located in the distal portion 322. The one or more markers 328 are formed of a material that increases visualization of the one or more markers 328 using imaging and is configured to aid a user in navigating the system 300 to a desired tissue site. In some embodiments, the one or more markers 328 are formed of a radiopaque material. In some embodiments, the one or more markers 328 are formed of a material that enhances echogenic properties of the one or more markers 328.

The tubular outer sheath 302 includes one or more openings 330 located in the distal portion 322. The one or more openings 330 allow for the perfusion of a fluid into or out of the sheath 302. Additionally, the one or more openings 330 prevent a vacuum effect causing the sheath 302 to stick to the dilator 314.

FIG. 3C is a side view of the transseptal access system 300 of FIG. 3A in a curved configuration during a transseptal crossing procedure, in accordance with embodiments of the disclosure. As illustrated in FIG. 3C, the system 300 includes a curved configuration having an inner radius 338 and an outer radius 340. In some embodiments, the curved configuration is achieved by the tubular outer sheath 302 having a pre-formed curve which imparts curvature to both the dilator 314 and the elongated puncturing device 310 to position the system 300 at a desired tissue location along the atrial septum 75. In some embodiments, the dilator 314 includes a pre-formed curve which imparts curvature to both the outer sheath 302 and the puncturing device 310. In other embodiments, a steering mechanism associated with the outer sheath 302 or the dilator 314 allows for the system 300 to achieve the curved configuration.

As illustrated in FIG. 3C, the leading edge 324 of the oblique profile 306 is aligned with the inner radius 338. In this configuration, as the system 300 achieves the curved configuration, the dilator 314 bends into the leading edge. This prevents space forming between the leading edge 324 and the dilator 314 and keeps the leading edge 324 snug against the dilator 314, reducing the likelihood of damage to tissue.

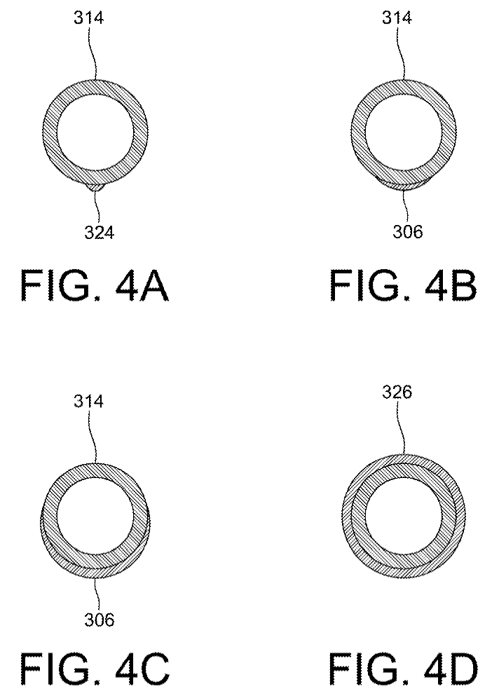

FIG. 5 is a side view of a transseptal access system 500 having a tubular outer sheath 502 with a distal end 504 including an oblique profile 506 including a partial linearly angled profile, in accordance with embodiments of the disclosure. The system 500 of FIG. 5 is identical to the system 300 of FIGS. 3A-3C, except for the shape of the oblique profile 506 as discussed below. As illustrated in FIG. 5, the oblique profile 506 comprises a partial linearly angled profile extending between a leading edge 524 of the distal end 504 and a proximal edge 526 of the distal end. Rather than having an apex, the leading edge 524 is perpendicular to a longitudinal axis 508 extending through the tubular outer sheath. The leading edge 524 is less than half of a circumference of the outer sheath 502. Extending from the leading edge 324 towards the proximal edge 526, the oblique profile 506 includes a linearly angled profile. The oblique profile 506 is configured such that a cross-sectional area of the distal end 504 in contact with a tissue increases as the tubular outer sheath 502 is advanced towards the tissue and through the tissue.

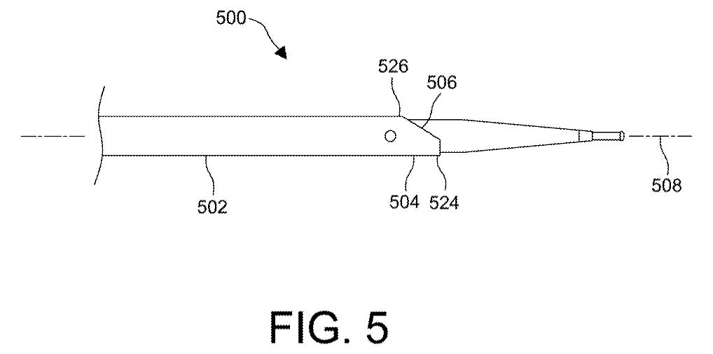

FIG. 6 is a side view of the transseptal access system 600 having a tubular outer sheath 602 with a distal end 604 including an oblique profile 606 including curved angled profile, in accordance with embodiments of the disclosure. The system 600 of FIG. 6 is identical to the system 300 of FIGS. 3A-3C, except for the shape of the oblique profile 506 as discussed below. As illustrated in FIG. 6, the oblique profile 606 comprises a curved profile extending between a leading edge 624 of the distal end 604 and a proximal edge 626 of the distal end 604. When viewed from the side, the oblique profile 606 does not appear linearly from the leading edge 624 to the proximal edge 626. Rather, the oblique profile 606 appears curved between the leading edge 624 and the proximal edge 626. The oblique profile 606 is configured such that a cross-sectional area of the distal end 604 in contact with a tissue increases as the tubular outer sheath 602 is advanced towards the tissue and through the tissue.

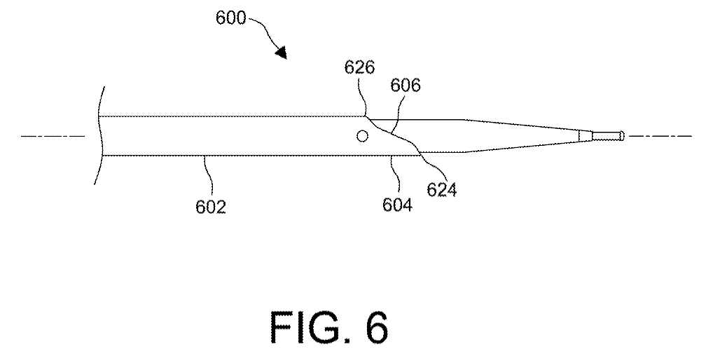

FIG. 7 is a side view of the transseptal access system 700 having a tubular outer sheath 702 with distal end 704 including an oblique profile 706 including a wave profile, in accordance with embodiments of the disclosure. As illustrated in FIG. 7, the oblique profile 706 comprises a wave profile including at least one crest 708 and at least one of trough 710. The at least one crest 710 forms a leading edge 724 of the distal end 704. The trough 710 forms a proximal edge 726 of the distal end 704. The oblique profile 706 is configured such that a cross-sectional area of the distal end 704 in contact with a tissue increases as the tubular outer sheath 702 is advanced towards the tissue and through the tissue.

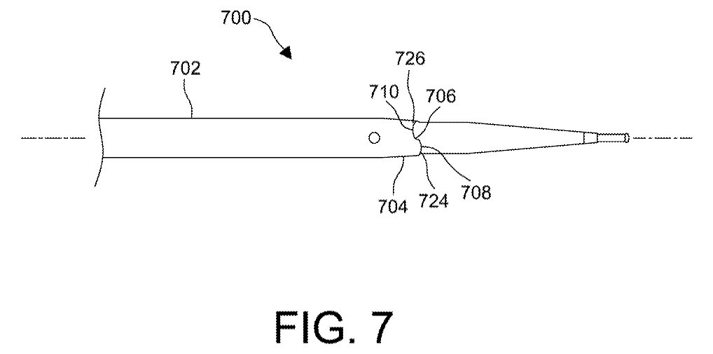

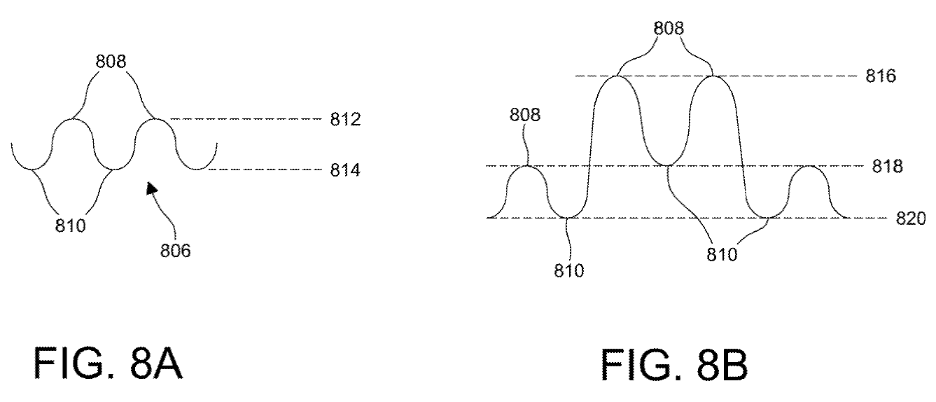

FIG. 8A illustrates a first arrangement for a wave profile, in accordance with embodiments of the disclosure. In FIG. 8A, the wave profile is shown unrolled rather than cylindrically. As illustrated, the wave profile includes a plurality of crests 808 and a plurality of troughs 810. The plurality of crests 808 are all aligned at a common longitudinal location 812. In this arrangement, a leading edge is formed at the common longitudinal location 812. Additionally, the plurality of troughs, 810 are aligned at a common longitudinal location 814 forming a proximal edge. As shown, the wave profile includes the plurality of crests 808 and the plurality of troughs 810 aligned at different longitudinal locations.

FIG. 8B illustrates a second arrangement for a wave profile, in accordance with embodiments of the disclosure. In FIG. 8B, the wave profile is shown unrolled rather than cylindrically. As illustrated, the wave profile includes a plurality of crests 808 and a plurality of troughs 810. Some of the plurality of crests 808 are aligned at a first common longitudinal location 816 forming a leading edge. Others of the plurality of crests 808 are aligned at a second common longitudinal location 818. As illustrated, at least one of the plurality of troughs 810 is aligned at the second common longitudinal location 818. Others of the plurality of troughs 810 are aligned at a third common longitudinal location 820 forming a proximal edge.

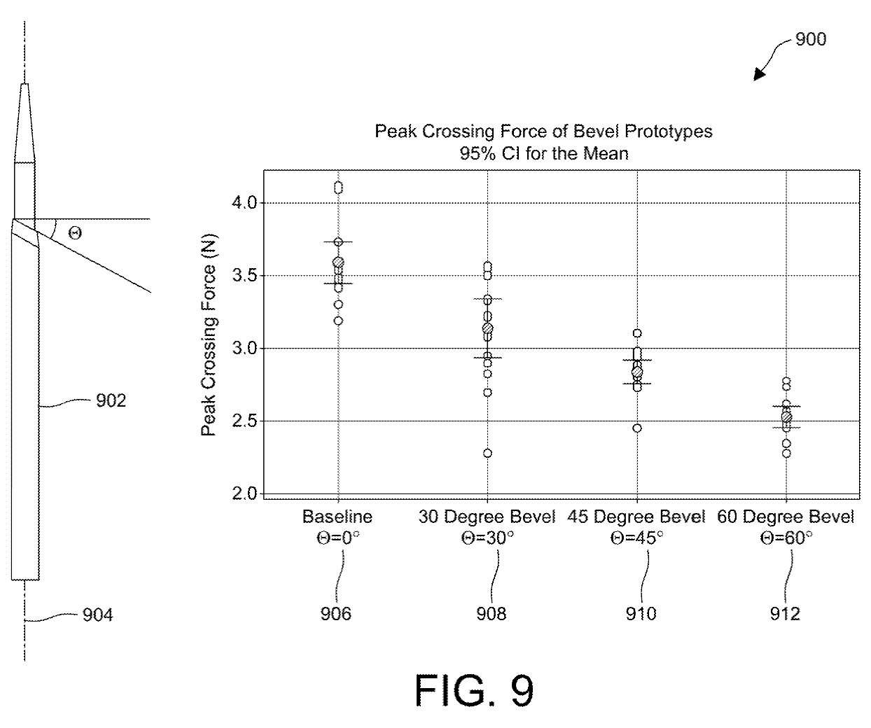

FIG. 9 illustrates peak crossing forces for various arrangements of sheaths with an end including an oblique profile having a linearly angled profile, in accordance with embodiments of the disclosure. In FIG. 9, the graph 900 illustrates a peak crossing force, measured in Newtons (N), required to pass a sheath 902 through a target tissue. A baseline 906 includes a sheath 902 having an end that is perpendicular to a longitudinal axis 904 passing through the center of the sheath 902. The baseline 906 sheath 902 has an angle Θ of zero. A sheath 902 having an oblique profile with an angle Θ of 30 degrees is shown at 908. A sheath 902 having an oblique profile with an angle Θ of 45 degrees is shown at 910. A sheath 902 having an oblique profile with an angle Θ of 60 degrees is shown at 912. As shown, the peak crossing force reduces as the angle Θ increases. This is because an increase in the angle Θ correlates to less cross-sectional area of the distal end in contact with the target tissue.

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps may be added or omitted without departing from the scope of this disclosure. Such steps may include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C. The terms “couples,” “coupled,” “connected,” “attached,” and the like along with variations thereof are used to include both arrangements wherein two or more components are in direct physical contact and arrangements wherein the two or more components are not in direct contact with each other (e.g., the components are “coupled” via at least a third component), but still cooperate or interact with each other.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. A system for use in a tissue crossing procedure, the system comprising:
an elongated puncturing device having a distal tip configured for puncturing tissue;
a dilator having a lumen configured for receiving the elongated puncturing device and a tapered distal portion configured to expand a puncture formed by the elongated puncturing device; and
a tubular outer sheath having a proximal portion, an opposite sheath distal portion, and a sheath lumen extending between the proximal portion and the opposite sheath distal portion, the sheath lumen configured for receiving the dilator, the sheath distal portion including a distal end having an oblique profile such that a cross-sectional area of the distal end in contact with the tissue increases as the sheath is advanced over the dilator towards the tissue and through the tissue.

2. The system of claim 1, wherein the oblique profile comprises a linearly angled profile extending between a leading edge of the distal end and a proximal edge of the distal end.

3. The system of claim 1, wherein the oblique profile comprises a curved profile extending between a leading edge of the distal end and a proximal edge of the distal end.

4. The system of claim 2, wherein the leading edge includes an apex, the proximal edge includes a vertex, and the apex and vertex are located on a common plane.

5. The system of claim 1, wherein the oblique profile comprises a partial linearly angled profile extending between a leading edge of the distal end and a proximal edge of the distal end.

6. The system of claim 5, wherein the leading edge is perpendicular to a longitudinal axis extending through the tubular outer sheath.

7. The system of claim 1, wherein the oblique profile comprises a wave profile.

8. The system of claim 7, wherein the wave profile includes at least one crest and at least one of trough.

9. The system of claim 8, wherein the at least one crest forms a leading edge of the distal end.

10. The system of claim 8, wherein the at least one crest and the at least one trough are aligned at a common longitudinal location.

11. The system of claim 8, wherein the at least one crest includes a plurality of crests and the at least one trough includes a plurality of troughs, and the plurality of crests or the plurality of troughs are aligned at different longitudinal locations.

12. The system of claim 1, wherein the tubular outer sheath includes one or more markers located in the sheath distal portion.

13. The system of claim 1, wherein the tubular outer sheath includes one or more openings located in the sheath distal portion.

14. The system of claim 1, wherein the system includes a curved configuration having an inner radius.

15. The system of claim 14, wherein a leading edge of the distal end is aligned with the inner radius such that the dilator bends into the leading edge.

16. A system for use in a tissue crossing procedure, the system comprising:
an elongated puncturing device having an energy emitting distal tip configured for puncturing tissue;
a dilator having a lumen configured for receiving the elongated puncturing device and a tapered distal portion configured to expand a puncture formed by the elongated puncturing device; and
a tubular outer sheath having a proximal portion, an opposite sheath distal portion, and a sheath lumen extending between the proximal portion and the opposite sheath distal portion, the sheath lumen configured for receiving the dilator, the sheath distal portion including a distal end having an oblique profile such that a cross-sectional area of the distal end in contact with the tissue increases as the sheath is advanced over the dilator towards the tissue and through the tissue.

17. The system of claim 16, wherein the system includes a curved configuration having an inner radius.

18. The system of claim 17, wherein a leading edge of the distal end is aligned with the inner radius such that the dilator bends into the leading edge.

19. The system of claim 16, wherein the oblique profile comprises a linearly angled profile, a curved profile, a wave profile, or a partial linear angled profile.

20. A system for use in a tissue crossing procedure, the system comprising:
an elongated puncturing device having a distal tip configured for puncturing tissue;
a dilator having a lumen configured for receiving the elongated puncturing device and a tapered distal portion configured to expand a puncture formed by the elongated puncturing device; and
a tubular outer sheath having a proximal portion, an opposite sheath distal portion, and a sheath lumen extending between the proximal portion and the opposite sheath distal portion, the sheath lumen configured for receiving the dilator, the sheath distal portion including a distal end having an oblique profile such that a cross-sectional area of the distal end in contact with the tissue increases as the sheath is advanced over the dilator towards the tissue and through the tissue;
wherein the system includes a curved configuration having an inner radius, and a leading edge of the distal end is aligned with the inner radius.
